(19) United States
(12) Patent Application Publication
(10) Pub. No.: US 2022/0323554 A1
(43) Pub. Date: Oct. 13, 2022

(54) POLYSACCHARIDE-PEPTIDE COMPLEX FOR LOWERING BLOOD SUGAR, BLOOD LIPID AND GLYCOSYLATED HEMOGLOBIN LEVELS, AND PREPARATION METHOD

(71) Applicants: Yubao LI, Huanggang (CN); Jingren HE, Wuhan (CN); CHINA YUNHONG HOLDINGS CO., LTD., Huanggang (CN)

(72) Inventors: Jingren HE, Wuhan (CN); Yubao LI, Huanggang (CN)

(21) Appl. No.: 17/728,965

(22) Filed: Apr. 25, 2022

Related U.S. Application Data
(63) Continuation of application No. PCT/CN2020/123563, filed on Oct. 26, 2020.

(30) Foreign Application Priority Data
Oct. 25, 2019 (CN) 201911020502.X

(51) Int. Cl. A61K 38/48; A23L 33/18; A23L 33/105; A23L 33/22; A23L 33/14; B01D 39/20; B01D 21/26; B01D 61/14; B01D 69/02; B01D 61/02; B01D 63/10; B01D 61/58; A61K 36/899; A61K 36/888; A61K 38/16; A61K 36/605; A61K 36/744; A61K 36/185; A61K 31/7004; A61K 36/076; A61K 36/734; A61K 36/06; A61K 38/47; A61K 38/46; A61P 3/06

(52) U.S. Cl. CPC A61K 38/4826; A23L 33/18; A23L 33/105; A23L 33/22; A23L 33/14; B01D 39/2068; B01D 39/2055; B01D 21/262; B01D 61/147; B01D 61/145; B01D 61/142; B01D 69/02; B01D 61/025; B01D 63/10; B01D 61/58; A61K 36/899; A61K 36/888; A61K 38/168; A61K 36/605; A61K 36/744; A61K 36/185; A61K 31/7004; A61K 36/076; A61K 36/734; A61K 36/06; A61K 38/47; A61K 38/465; A61P 3/06; A23V 2002/00; B01D 2325/34; B01D 2325/02

(57) ABSTRACT

Disclosed are a polysaccharide-peptide composite and a method of preparing the same. The polysaccharide-peptide composite is prepared from a bitter melon peptide (BMP) powder, gardenia fruit oil, a soybean polypeptide powder, an oat dietary fiber powder, a konjac powder, a corn silk, a mulberry leaf extract, a Poria cocos extract, a hawthorn extract, nutritional yeast and a pancreatin. The BMP powder is prepared by temperature-controlled hydrolysis, staged enzymatic hydrolysis and multiple filtrations. The gardenia fruit oil is prepared by staged enzymatic hydrolysis, multi-step centrifugation, filtration and stratification.

### CROSS-REFERENCE TO RELATED APPLICATIONS

This application is a continuation of International Patent Application No. PCT/CN2020/123563, filed on Oct. 26, 2020, which claims the benefit of priority from Chinese Patent Application No. 201911020502.X, filed on Oct. 25, 2019. The content of the aforementioned application, including any intervening amendments thereto, is incorporated herein by reference in its entirety.

### TECHNICAL FIELD

This application relates to nutritional functional foods and biological fermentation, and more particularly to a polysaccharide-peptide composite for lowering blood sugar, blood lipid, and glycated hemoglobin and a method of preparing the same.

### BACKGROUND

In recent years, human life has been improved gradually, accompanied by the ascending incidence of diseases, such as diabetes, hyperlipidemia, and obesity, in elder people as well as younger people. Although in recent years, traditional Chinese medicine, western medicine, and health food aiming to prevent the above diseases have been available on the market, there are still many difficult problems needed to be solved. As a consequence, there is still a need for researchers to constantly develop new drugs and healthy foods for the prevention and treatment of diabetes, hyperlipidemia, and obesity.

Blood sugar and glycated hemoglobin are important indicators for evaluating the degree of diabetes, hyperlipidemia, and obesity. The blood sugar is a monosaccharide in the blood that is derived from carbohydrates in food, and usually merely refers to glucose. The blood sugar test results reveal the real-time blood sugar level. Glycated hemoglobin (HbA1c) is formed through non-enzymatic reaction between blood glucose and the N-terminal valine of hemoglobin in red blood cells via the cell membrane. The synthesis rate of HbA1c is proportional to the sugar concentration in the environment where the red blood cells are located. HbA1c is a product of the combination of hemoglobin in red blood cells and blood sugar. The combination of hemoglobin and blood sugar is irreversible, and will remain for about 120 days. Therefore, compared with blood sugar detection, the HbA1c detection is more clinically significant, which is known as the “gold standard” for diabetes monitoring, and thus often used as an indicator of the daily blood glucose control in clinical testing.

Hence, it is particularly important to provide a kind of food and drug that can more effectively lower the level of blood sugar and glycated hemoglobin.

### SUMMARY

To overcome the above deficiencies in the prior art, the present disclosure provides a polysaccharide-peptide composite for lowering blood sugar, blood lipid, and glycated hemoglobin and a method of preparing same. The method provided herein adopts a special extraction process for increasing the content of bitter melon polypeptide, combined with the use of other natural plant components to better lowering blood sugar and glycated hemoglobin.

Technical solutions of the present disclosure are described as follows.

In a first aspect, this application provides a polysaccharide-peptide composite, which is prepared from 20-25 parts by weight of oat dietary fiber powder, 10-15 parts by weight of konjac powder, 10-15 parts by weight of a corn silk, 20-30 parts by weight of bitter melon peptide (BMP) powder, 10-12 parts by weight of soybean polypeptide powder, 5-10 parts by weight of a mulberry leaf extract, 5-10 parts by weight of a gardenia fruit oil, 5-10 parts by weight of cocoa powder, 5-10 parts by weight of L-arabinose, 3-5 parts by weight of a Poria cocos extract, 5-10 parts by weight of a hawthorn extract, 1-2 parts by weight of nutritional yeast, 2-5 parts by weight of a pancreatin, and 5-8 parts by weight of xylitol.

In some embodiments, the nutritional yeast comprises selenium-rich yeast with a selenium concentration of 3000 ppm and chromium-rich yeast with a chromium concentration of 2000 ppm, with a weight ratio of 1:2.

In some embodiments, the pancreatin comprises trypsin, amylopsin and pancreatic lipase with a weight ratio of 1:2:2.

In some embodiments, the BMP powder is prepared through steps of:
(S11) soaking a raw material with deionized water at 25° C. for 10-12 h, wherein a weight ratio of the raw material to the deionized water is 1:5, and the raw material is fresh bitter melon, dried bitter melon, bitter melon seeds, or a combination thereof; and taking the raw material out followed by rinsing with deionized water 2-3 times;
(S2) subjecting the raw material to drying, crushing and grinding to obtain a bitter melon slurry;
(S3) mixing the bitter melon slurry with a buffer solution in a weight ratio of 1:(3-5) to form a mixed system; recording a volume of the mixed system as initial volume V₀; and regulating the mixed system to pH 6.8-7 followed by temperature treatment to obtain a bitter melon extract;
wherein the temperature treatment is performed through steps of:
(S31) heating the mixed system to 45-55° C. and keeping the mixed system at 45-55° C. for 45-60 min; cooling the mixed system to 20-25° C., and keeping the mixed system at 20-25° C. for 25-30 min; and recording a volume of the mixed system at this time as a first volume V₁;
(S32) adding a first mixed liquid to the mixed system, wherein the first mixed liquid comprises deionized water and the buffer solution with a weight ratio of 4:1, and a volume of the first mixed liquid is calculated by: (V₀-V₁)*0.6; heating the mixed system to 60-75° C., and keeping the mixed system at 60-75° C. for 60-75 min; cooling the mixed system to 45-55° C., and keeping the mixed system at 45-55° C. for 30-35 min; and recording a volume of the mixed system at this time as second volume V₂; and
(S33) adding a second mixed liquid to the mixed system, wherein second mixed liquid comprises deionized water and the buffer solution with a weight ratio of 3:1, and a volume of the second mixed liquid is calculated by: (V₀-V₂)*0.75; heating the mixed system to 80-90° C., and keeping the mixed system at 80-90° C. for 75-85 min; and cooling the mixed system to 60-75° C. and keeping the mixed system at 60-75° C. for 35-45 min;
(S4) cooling the bitter melon extract to 20-25° C. followed by enzymatic hydrolysis to obtain an enzymatic hydrolysis product, wherein the enzymatic hydrolysis is performed through steps of:
regulating the bitter melon extract to pH 7.5-8.5; adding trypsin to the bitter melon extract followed by heating to 35-40° C. under stirring at 80-100 rpm and keeping at 35-40° C. for 45-60 min to obtain a first enzymatic hydrolysis system, wherein the trypsin is 5% by weight of the bitter melon extract;
cooling the first enzymatic hydrolysis system to 20-25° C., and adjusting the first enzymatic hydrolysis system to pH 3.0-4.0; adding pectinase to the first enzymatic hydrolysis system followed by heating to 45-55° C. under stirring at 80-100 rpm and keeping at 45-55° C. for 40-60 min to obtain a second enzymatic hydrolysis system, wherein the pectinase is 3% by weight of the first enzymatic hydrolysis system; and
cooling the second enzymatic hydrolysis system to 20-25° C., and regulating the second enzymatic hydrolysis system to pH 4.5-5.0; adding a cellulase to the second enzymatic hydrolysis system followed by heating to 55-60° C. under stirring at 80-100 rpm and keeping at 55-60° C. for 30-45 min to obtain the enzymatic hydrolysis product, wherein the cellulase is 2% by weight of the second enzymatic hydrolysis system;
(S5) heating the enzymatic hydrolysis product to 90° C. followed by keeping at 90° C. for 10 min for inactivation, so as to obtain a crude BMP extraction system; adding activated carbon to the crude BMP extraction system followed by uniform stirring, wherein the activated carbon is 4-5% by weight of the crude BMP extraction system; and keeping the crude BMP extraction system at 65° C. for 60-90 min followed by centrifugation to obtain a first supernatant; and
filtering the first supernatant with diatomite at 0.2-0.3 MPa to obtain a first filtrate; and adding activated carbon to the first filtrate followed by standing for 45-50 min and centrifugation to obtain a second supernatant, wherein the activated carbon is 4-5% by weight of the first filtrate;
(S6) filtering the second supernatant with a ceramic microfiltration membrane with a pore size of 0.5-0.8 m at 55-65° C. to obtain a second filtrate;
filtering the second filtrate with a spiral wound ultrafiltration membrane having a cut-off molecular weight of 100-200 kDa at 45-50° C. to obtain a third filtrate; and
concentrating the third filtrate with a spiral-wound reverse-osmosis membrane at 35-40° C. to remove water, residual inorganic salts and small molecule impurities to obtain a BMP concentrate; and
(S7) subjecting the BMP concentrate to vacuum freeze drying to obtain the BMP powder, wherein the BMP powder comprises 30% or more by weight of BMP.

In some embodiments, the buffer solution is a phosphate buffer solution prepared from disodium hydrogen phosphate dodecahydrate and sodium dihydrogen phosphate dihydrate.

In some embodiments, the gardenia fruit oil is extracted through steps of:
(S1) soaking a fresh gardenia fruit in water at 25° C. for 24-36 h, followed by rinsing with water 2-3 times, drying, grinding, screening with a 100-mesh sieve to obtain a gardenia fruit powder;
(S2) mixing the gardenia fruit powder with deionized water in a weight ratio of 1:(5-10) to obtain a mixture; and subjecting the mixture to enzymatic hydrolysis to obtain an enzymatic hydrolysis product, wherein the enzymatic hydrolysis is performed through steps of:
adding trypsin and a permeability-regulating fluid to the mixture, wherein the trypsin is 5% by weight of the gardenia fruit powder, and the permeability-regulating fluid is 45-55% by weight of the gardenia fruit powder; and adjusting the mixture to pH 6.5-7.5, followed by heating to 42-45° C. under stirring, and keeping at 42-45° C. for 30-45 min to obtain a first enzymatic hydrolysis system, wherein the permeability-regulating fluid consists of an acid solution, glycerol, sodium chloride, and lysozyme in a weight ratio of 1:(0.7-1.0):(0.02-0.05):(0.03-0.06);
cooling the first enzymatic hydrolysis system to 20-25° C., regulating the first enzymatic hydrolysis system to pH 3.5-4.5; and adding pectinase to the first enzymatic hydrolysis system followed by heating to 50-60° C. under stirring, and keeping at 50-60° C. for 30-35 min to obtain a second enzymatic hydrolysis system, wherein the pectinase is 4% by weight of the first enzymatic hydrolysis system; and
cooling the second enzymatic hydrolysis system to 20-25° C., and regulating the second enzymatic hydrolysis system to pH 4.0-5.5; and adding cellulase to the second enzymatic hydrolysis system followed by heating to 50-65° C. under stirring, and keeping at 50-65° C. for 25-35 min to obtain the enzymatic hydrolysis product, wherein the cellulase is 3.5% by weight of the second enzymatic hydrolysis system;
(S3) heating the enzymatic hydrolysis product to 85° C. followed by keeping at 85° C. for 10 min for inactivation to obtain a gardenia fruit extraction system; and
(S4) adding activated carbon to the gardenia fruit extraction system followed by uniform stirring, keeping at 65° C. for 65-85 min and centrifugation to obtain a crude gardenia fruit oil extract, wherein the activated carbon is 3% by weight of the gardenia fruit extraction system; filtering the crude gardenia fruit oil extract with diatomite at 0.3-0.4 MPa to obtain a filtrate; adding activated carbon to the filtrate followed by standing for 45-50 min and centrifugation to obtain a supernatant, wherein the activated carbon is 3% by weight of the filtrate; and subjecting the supernatant to standing for 2-3 h to collect an oil layer as the gardenia fruit oil.

In a second aspect, this application also provides a method of preparing the polysaccharide-peptide composite, comprising:
(S1) preparing the BMP powder, the gardenia fruit oil, the Poria cocos extract, the mulberry leaf extract and the hawthorn extract, wherein the Poria cocos extract, the mulberry leaf extract or the hawthorn extract is prepared through steps of:
soaking a raw material in water for 12-15 h followed by boiling for 1-2 h and filtration to obtain a first filtrate and a first filter residue, wherein a weight ratio of the raw material to the water is 1:(8-10);
drying the first filter residue; soaking a dried first filter residue with a 60% (v/v) ethanol solution for 1-2 h followed by heating to 65-75° C., extraction for 1.5-2 h, standing at 8° C. for 24 h, and filtration to obtain a second filtrate and a second filter residue, wherein a weight ratio of the dried first filter residue to the ethanol solution is 1:(6-8); and during the extraction, stirring is performed once every 10 min with a stirring rate of 200-300 rpm; and
soaking the second filter residue with a 60% (v/v) ethanol solution for 6-7 h followed by heating to 65-75° C., extraction for 2.5-3 h, standing at 8° C. for 24 h, and filtration to obtain a third filtrate and a third filter residue, wherein a weight ratio of the second filter residue to the ethanol solution is 1:(8-10); and during the extraction, stirring is performed once every 10 min with a stirring rate of 200-300 r/min; and combining the first filtrate, the second filtrate, and the third filtrate to obtain the Poria cocos extract, the mulberry leaf extract, or the hawthorn extract;
(S2) uniformly mixing 20-25 parts by weight of the oat dietary fiber powder, 10-15 parts by weight of the konjac powder, 10-15 parts by weight of the corn silk, 20-30 parts by weight of the BMP powder, 10-12 parts by weight of the soybean polypeptide powder, 5-10 parts by weight of the mulberry leaf extract, 5-10 parts by weight of the gardenia fruit oil, 5-10 parts by weight of the cocoa powder, 3-5 parts by weight of the Poria cocos extract, and 5-10 parts by weight of the hawthorn extract to obtain a mixture; mixing the mixture with deionized water in a weight ratio of 1:(5-8) followed by stirring, heating to 45-65° C., and vacuum concentration to obtain a concentrate; and
(S3) mixing the concentrate with 5-10 parts by weight of the L-arabinose, 1-2 parts by weight of the nutritional yeast, 2-5 parts by weight of the pancreatin, and 5-8 parts by weight of the xylitol followed by stirring to obtain the polysaccharide-peptide composite.

This application has at least the following beneficial effects.

Through the extraction process including periodic warming, repeated digestion and multiple filtering, the content of BMP-protein in the BMP is equal to or greater than 30%. Moreover, the BMP is used in conjunction with other components, showing a significant effect in view of lowering blood sugar and glycated hemoglobin, and allowing users to get rid of side effects brought by the use of chemical hypoglycemic drugs.

Other advantages, objectives, and characteristics of the present application will be partly revealed by the following description and partly understood by those skilled in the art through the study and practice of the present application.

### DETAILED DESCRIPTION OF EMBODIMENTS

The present application will be further described in detail below to allow those skilled in the art to implement it according to the specification.

It should be understood that the terms used herein, such as “has”, “include” and “comprise”, do not match the existence or addition of one or more other components or the combination thereof.

It should be noted that if there are no special instructions, the following test method described in the examples are conventional methods, and described reagents and materials can be available commercially.

### Example 1

Provided herein was a polysaccharide-peptide composite, which was prepared from 20 parts by weight of oat dietary fiber powder, 11 parts by weight of konjac powder, 12 parts by weight of a corn silk, 20 parts by weight of bitter melon polypeptide (BMP) powder, 10 parts by weight of soybean polypeptide powder, 6 parts by weight of a mulberry leaf extract, 6 parts by weight of a gardenia fruit oil, 5 parts by weight of cocoa powder, 5 parts by weight of L-arabinose, 3 parts by weight of a Poria cocos extract, 6 parts by weight of a hawthorn extract, 1 parts by weight of nutritional yeast, 2 parts by weight of a pancreatin, and 5 parts by weight of xylitol. The nutritional yeast included selenium-rich yeast with a selenium concentration of 3000 ppm and chromium-rich yeast with a chromium concentration of 2000 ppm. The weight ratio of the selenium-rich yeast to the chromium-rich yeast was 1:2. The pancreatin included trypsin, amylopsin and pancreatic lipase with a weight ratio of 1:2:2.

The BMP powder was prepared through the following steps.
(S1) A raw material was soaked in deionized water at 25° C. for 10-12 h, preferably, 10.5 h, where a weight ratio of the raw material to the deionized water was 1:5, and the raw material was a fresh bitter melon, a dried bitter melon, bitter melon seeds, or a combination thereof. After that, the raw material was taken out followed by rinsing with deionized water 2-3 times to remove pesticide residues, drug residues and impurities to obtain a rinsed raw material.
(S2) The rinsed material was subjected to drying, crushing and grinding to obtain a bitter melon slurry.
(S3) The bitter melon slurry was mixed with a buffer solution in a weight ratio of 1:(3-5), preferably, 1:4, to form a mixed system, where the buffer solution was a buffer system containing acid, alkali, salt and other reagents, i.e., phosphate buffer. The volume of the mixed system was recorded as initial volume V₀. Then the mixed system was regulated to pH 6.8-7 followed by temperature treatment to obtain a bitter melon extract.

The temperature treatment was performed through the following steps.

The mixed system was heated to 45-55° C. (preferably, 50° C.), kept for 45-60 min (preferably, 55 min), cooled to 20-25° C. (preferably, 22° C.), and kept for 25-30 min (preferably, 28 min), and a volume of the mixed system was recorded at this time as a first volume V₁. During the aforementioned heating and insulation processes, the water and acids in the mixed system were evaporated, resulting in the change in the solubility of acid, base and inorganic ions in the mixed system. Therefore, after the aforementioned heating and insulation processes, a first mixed liquid was added to the mixed system for compensation such that the mixed system could constantly stay at a good extraction environment, where the first mixed liquid contained deionized water and the buffer solution with a weight ratio of 4:1; and a volume of the first mixed liquid was calculated by: (V₀-V₁)*0.6. After being added with the first mixed liquid, the mixed system was heated to 60-75° C. (preferably, 65° C.), kept at 60-75° C. for 60-75 min (preferably, 65 min), cooled to 45-55° C. (preferably, 50° C.), and kept at 45-55° C. for 30-35 min (preferably, 32 min), and a volume of the mixed system was recorded at this time as a second volume V₂. A second mixed liquid was added to the mixed system, where the second mixed liquid contained deionized water and the buffer solution with a weight ratio of 3:1; and a volume of the second mixed liquid was calculated by: (Vₜ-V₂)*0.75. In this case, the temperature of heating and insulation was higher, so the evaporation of water and acids in the mixed system was greater. As a consequence, the addition volume of the second mixed liquid was increased (to 75%), and the proportion of the buffer solution in the second mixed liquid was also increased. After being added with the second mixed liquid, the mixed system was heated to 80-90° C. (preferably, 85° C.), kept at 80-90° C. for 75-85 min (preferably, 80 min), cooled to 60-75° C. (preferably, 70° C.), and kept at 60-75° C. for 35-45 min (preferably, 40 min).

In this step, through periodic heating and insulation, cellular structure (such as cell wall) of components were repeatedly impacted and destroyed under different temperature environment. And at the same time, after heating and insulation, the reaction system was supplemented with corresponding proportion of water and the buffer solution for compensating the evaporation of water and acids, so that the reaction system was always in a great extraction environment to achieve the best extraction effect.
(S4) After the bitter melon extract was cooled to 20-25° C., enzymatic hydrolysis was performed on the bitter melon extract to obtain an enzymatic hydrolysis product, where the enzymatic hydrolysis was performed through the following steps.

Primary Enzymatic Hydrolysis

The bitter melon extract was regulated to pH 7.5-8.5 (preferably, 7.0). Trypsin was added to the extract followed by heating to 35-40° C. (preferably, 37° C.) under stirring at 80-100 r/min and keeping at 35-40° C. for 45-60 min (preferably, 55 min) to obtain a first enzymatic hydrolysis system, where the trypsin was 5% by weight of the bitter melon extract.

Secondary Enzymatic Hydrolysis

After the first enzymatic hydrolysis system was cooled to 20-25° C., the first enzymatic hydrolysis system was regulated to pH 3.0-4.0 (preferably, 3.5). Pectinase was added to the first enzymatic hydrolysis system followed by heating to 45-55° C. (preferably, 50° C.) under stirring at 80-100 r/min and keeping at 45-55° C. for 40-60 min (preferably, 50 min) to obtain a second enzymatic hydrolysis system, where the pectinase was 3% by weight of the first enzymatic hydrolysis system.

Third Enzymatic Hydrolysis

After the second enzymatic hydrolysis system was cooled to 20-25° C., the second enzymatic hydrolysis system was regulated to pH 4.5-5.0 (preferably, 4.7). Cellulase was added to the second enzymatic hydrolysis system followed by heating to 55-60° C. (preferably, 58° C.) under stirring at 80-100 r/min and keeping at 55-60° C. for 30-45 min (preferably, 35 min) to obtain the enzymatic hydrolysis product, where the cellulase was 2% by weight of the second enzymatic hydrolysis system.

As the components used herein were plant components, of which cellular structure contained cell walls. Therefore, in this step, through the use of different enzymes and enzymatic conditions under different stages for complete enzymatic hydrolysis of cell walls, cellulose and pectin of the cell walls were destroyed, which facilitated active ingredients (such as BMP protein) in the cell walls to release fully to enhance the extraction efficiency.
(S5) After the enzymatic hydrolysis was completed, the enzymatic hydrolysis product was subjected to heating to 90° C. for 10 min for inactivation to obtain a crude BMP extraction system. Activated carbon was added to the crude BMP extraction system followed by uniform stirring, keeping at 65° C. for 60-90 min (preferably, 75 min), centrifugation, and residue removal to obtain a first supernatant, where the activated carbon was 4-5% by weight of the crude BMP extraction system.

The first supernatant was filtered with diatomite at 0.2-0.3 MPa (preferably, 0.25 MPa) to obtain a first filtrate. The first filtrate was added with activated carbon followed by standing for 45-50 min, centrifugation, and residue removal to obtain a second supernatant, where the activated carbon was 4-5% by weight of the first filtrate.

Through the adsorption treatment by activated carbon and diatomite, the pigment, suspended particles and colloid in the enzymatic hydrolysate of BMP were removed to offer a final product with high purity.
(S6) The second supernatant was filtered with a ceramic microfiltration membrane with a pore size of 0.5-0.8 m at 55-65° C. (preferably, 60° C.) to obtain a second filtrate, where the ceramic microfiltration membrane was used in parallel with three membranes.

The second filtrate was filtered with a spiral wound ultrafiltration membrane having a cut-off molecular weight of 100-200 kDa at 45-50° C. (preferably, 48° C.) to remove macromolecular impurities to obtain a third filtrate, where the spiral wound ultrafiltration membrane was used in parallel with two membranes.

The third filtrate was concentrated with a spiral reverse-osmosis membrane with having a cut-off molecular weight of 150-1000 kDa below 40° C. to remove water, residual inorganic salts and small molecule impurities to obtain a BMP concentrate, where the solid content of the BMP concentrate was equal to or more than 40%; and the spiral reverse-osmosis membrane was a high-pressure concentration membrane, made of polysulfone (PS), polyethersulfone (PFS), or other composite material film, and used in parallel with four membranes.

In this step, the multilayer membrane separation and purification technology was used to separate and purify the BMP protein. The low concentration temperature used herein effectively guaranteed the natural activity and high content of the BMP protein.
(S7) The BMP concentrate was subjected to vacuum freeze drying to obtain the BMP powder, where the BMP powder contained 30% or more by weight of BMP.

Moreover, gardenia fruit was abundant, cheap, and had a variety of functions, and has played a more and more important role in the modern food industry. Specifically, gardenia fruit mainly contained flavonoids, iridoids, crocin, pectin, tannins, polysaccharides, crocetin, and volatile oil. Among them, flavonoids had an auxiliary therapeutic effect on hypertension and other diseases, and could reduce blood pressure and blood sugar. Hence, this example also provided a method for extracting the gardenia fruit oil, which included the following steps.
(S1) A fresh gardenia fruit was soaked in water at 25° C. for 24-36 h, followed by rinsing with water 2-3 times, drying, grinding, screening with a 100-mesh sieve to obtain a gardenia fruit powder.
(S2) The gardenia fruit powder was mixed with deionized water in a weight ratio of 1:(5-10) (preferably, 8) to obtain a mixture, and the mixture was subjected to enzymatic hydrolysis to obtain an enzymatic hydrolysis product, where the enzymatic hydrolysis was performed through the following steps.

Primary Enzymatic Hydrolysis

The mixture was added with trypsin and a permeability-regulating fluid, where the trypsin was 5% by weight of the gardenia fruit powder; and the permeability-regulating fluid was 45-55% (preferably, 50%) by weight of the gardenia fruit powder. The mixture was regulated to pH 6.5-7.5 (preferably, 7.0), followed by heating to 42-45° C. (preferably, 43.5° C.) under stirring, and keeping at 42-45° C. for 30-45 min (preferably, 35 min) to obtain a first enzymatic hydrolysis system, where the permeability-regulating fluid consisted of an acid solution, glycerol, sodium chloride, and lysozyme in a weight ratio of 1:(0.7-1.0):(0.02-0.05):(0.03-0.06), preferably, 1:0.8:0.03:0.04; and the acid solution was a citric acid-sodium citrate buffer solution with a pH of 6-7.

Secondary Enzymatic Hydrolysis

After the first enzymatic hydrolysis system was cooled to 20-25° C., the first enzymatic hydrolysis system was regulated to pH 3.5-4.5 (preferably, 4.0). Pectinase was added to the first enzymatic hydrolysis system, followed by heating to 50-60° C. (preferably, 55° C.) under stirring, and keeping at 50-60° C. for 30-35 min (preferably, 32 min) to obtain a second enzymatic hydrolysis system, where the pectinase was 4% by weight of the first enzymatic hydrolysis system.

Tertiary Enzymatic Hydrolysis

After the second enzymatic hydrolysis system was cooled to 20-25° C., the second enzymatic hydrolysis system was regulated to pH 4.0-5.5 (preferably, 5.0). Cellulase was added to the second enzymatic hydrolysis system, followed by heating to 50-65° C. (preferably, 60° C.) under stirring, and keeping at 50-65° C. for 25-35 min (preferably, 30 min) to obtain the enzymatic hydrolysis product, where the cellulase was 3.5% by weight of the second enzymatic hydrolysis system.
(S3) After the enzymatic hydrolysis was completed, the third enzymatic hydrolysis product was heated to 85° C. followed by keeping at 85° C. for 10 min for inactivation to obtain a gardenia fruit extraction enzymatic hydrolysis system.
(S4) The gardenia fruit enzymatic hydrolysis system was added with activated carbon followed by uniform stirring, keeping at 65° C. for 65-85 min (preferably, 75 min), centrifugation, and residue removal to obtain a crude gardenia fruit oil extract, where the activated carbon was 4-5% by weight of the gardenia fruit enzymatic hydrolysis system. Then the crude gardenia fruit oil extract was filtered with diatomite at 0.3-0.4 MPa (preferably, 0.35 MPa) to obtain a filtrate. After that, the filtrate was added with activated carbon followed by standing for 45-50 min, centrifugation, and residue removal to obtain a supernatant, where the activated carbon was 3% by weight of the filtrate. The supernatant was subjected to standing for 2-3 h to collect an oil layer as the gardenia fruit oil.

### Example 2

Example 2 was different from Example 1 in the composition of the polysaccharide-peptide composite. In Example 2, the polysaccharide-peptide composite was prepared from 25 parts by weight of oat dietary fiber powder, 15 parts by weight of konjac powder, 14 parts by weight of a corn silk, 28 parts by weight of BMP powder, 11 parts by weight of soybean polypeptide powder, 9 parts by weight of a mulberry leaf extract, 8 parts by weight of a gardenia fruit oil, 9 parts by weight of cocoa powder, 10 parts by weight of L-arabinose, 5 parts by weight of a Poria cocos extract, 8 parts by weight of a hawthorn extract, 2 parts by weight of nutritional yeast, 4.5 parts by weight of pancreatin, 7.5 parts by weight of xylitol, and 200 parts by weight of water.

### Example 3

Example 3 was different from Example 1 in the composition of the polysaccharide-peptide composite. In Example 3, the polysaccharide-peptide composite was prepared from 22 parts by weight of oat dietary fiber powder, 13 parts by weight of konjac powder, 12.5 parts by weight of a corn silk, 25 parts by weight of a BMP powder, 11 parts by weight of soybean polypeptide powder, 8 parts by weight of a mulberry leaf extract, 8 parts by weight of a gardenia fruit oil, 8 parts by weight of cocoa powder, 7 parts by weight of L-arabinose, 4 parts by weight of a Poria cocos extract, 8 parts by weight of a hawthorn extract, 1.5 parts by weight of a nutritional yeast, 3.5 parts by weight of a pancreatin, 6.5 parts by weight of xylitol, and 200 parts by weight of water.

Molecular weight detection results of BMP were as follows.

The BMP of Comparative Example 1 was prepared according to Example 1 of Chinese Patent Application No. 201710832199.8. The BMP samples of Examples 1-3 and Comparative Example 1 were detected by using a high-performance gel filtration chromatography to acquire the molecular weight of BMP and distribution range thereof. The results were shown in Table 1.

TABLE 1
Molecular weight of BMP and distribution range thereof

| | Percentage of peak | | | | Number-average | | | | Weight-average | | | |
|---|---|---|---|---|---|---|---|---|---|---|---|---|
| | area (%, λ 200 nm) | | | | molecular weight | | | | molecular weight | | | |
| Range of | Compar- | | | | Compar- | | | | Compar- | | | |
| molecular | ative | | | | ative | | | | ative | | | |
| weight | Exam- | Exam- | Exam- | Exam- | Exam- | Exam- | Exam- | Exam- | Exam- | Exam- | Exam- | Exam- |
| (Da) | ple 1 | ple 1 | ple 2 | ple 3 | ple 1 | ple 1 | ple 2 | ple 3 | ple 1 | ple 1 | ple 2 | ple 3 |
| >7000 | 16.35 | 9.45 | 9.79 | 9.86 | 9623 | 8131 | 8268 | 8311 | 9799 | 8199 | 8615 | 8562 |
| 7000-5000 | 22.66 | 32.64 | 33.52 | 33.69 | 6721 | 6215 | 6452 | 6141 | 6878 | 6477 | 6547 | 6458 |
| 5000-3000 | 14.32 | 19.31 | 19.99 | 20.58 | 4520 | 4202 | 4321 | 4158 | 4625 | 4325 | 4475 | 4341 |
| 3000-1000 | 21.47 | 25.91 | 23.43 | 25.01 | 1652 | 2852 | 2745 | 2902 | 2978 | 2752 | 2872 | 2958 |
| <1000 | 25.20 | 12.69 | 13.27 | 10.86 | 879 | 756 | 744 | 698 | 903 | 812 | 805 | 789 |

In the method for preparing the BMP provided herein, through periodic heating and insulation, cellular structure (such as cell wall) of components were repeatedly impacted and destroyed under different temperature environments. And at the same time, after heating and insulation, the reaction system was supplemented with a corresponding proportion of water and the buffer solution for compensating the evaporation of water and acids, so that the reaction system was always in a great extraction environment. Moreover, through further periodic heating, repeated enzymatic hydrolysis, and multilayer membrane separation and purification technology, the BMP protein in the obtained BMP powder was greater than 30%. In addition, there was no soybean polypeptide protein and other non-BMP proteins in the obtained extracts. As can be seen from Table 1, the BMP fragment within the range of 5000-7000 Da prepared herein was close to 30%, which was exactly the fragment with the function of regulating blood sugar and the fragment size closest to the molecular weight of insulin. Therefore, the obtained BMP extract had a good effect on regulating blood sugar metabolism, especially could greatly improve the binding ability of insulin receptors and lowering blood sugar.

Detection results of gardenia fruit oil were as follows.

The gardenia fruit oil of Comparative Example 2 was prepared according to Example 1 of Chinese Patent Application No. 201110321487.X. The gardenia fruit oil samples of Examples 1-3 and Comparative Example 2 were detected to acquire the content of crocin, chlorogenic acid, flavone and geniposide. Those components were mainly effective components for lowering blood sugar and blood lipid. The results were shown in Table 2.

TABLE 2
Content of crocin, chlorogenic acid, flavone
and geniposide in the gardenia fruit oil

| | Crocin | Flavone | Chlorogenic | Geniposide |
|---|---|---|---|---|
| | (mg/g) | (mg/g) | acid (mg/g) | (mg/g) |
| Comparative | 1.24 ± 0.31 | 3.51 ± 0.25 | 3.14 ± 0.25 | 3.42 ± 0.37 |
| Example 2 | | | | |
| Example 1 | 2.24 ± 0.41 | 6.58 ± 0.44 | 4.15 ± 0.58 | 3.92 ± 0.21 |
| Example 2 | 2.39 ± 0.29 | 7.25 ± 0.38 | 4.21 ± 0.13 | 3.82 ± 0.35 |
| Example 3 | 2.18 ± 0.11 | 6.95 ± 0.57 | 4.23 ± 0.34 | 4.12 ± 0.50 |

In the method provided herein, through the use of different enzymes and enzymatic conditions under different stages for complete enzymatic hydrolysis of cell walls of the gardenia fruit, cellulose and pectin of the cell walls were destroyed. And at the same time, acid solutions, glycerol, sodium chloride, and lysozyme could alter the permeability of cell wall or cell membrane by altering their structure. Therefore, by employing a permeability regulatory solution to regulate the permeability of cell membrane and/or cell wall, the structure of cell membrane and/or cell wall could be destroyed, which facilitated active ingredients (such as crocin, and flavone) to release fully to fulfil the hypoglycemic effect.

### Example 4

Provided was a method of preparing the polysaccharide-peptide composite, which included the following steps.
(S1) The BMP powder, the gardenia fruit oil, the Poria cocos extract were prepared according to any one of Example 1-3. The mulberry leaf extract and the hawthorn extract were prepared by using the following method, which was described in detail below.

A raw material (Poria cocos, mulberry leaf or hawthorn) was soaked in water for 12-15 h (preferably, 13 h), heated to boiling for 1-2 h, and filtrated to obtain a first filtrate and a first filter residue, where a weight ratio of the raw material to the water was 1:(8-10).

The first filter residue was dried, soaked with a 60% (v/v) ethanol solution with for 1-2 h (preferably, 1.5 h) followed by heating to 65-75° C. (preferably, 70° C.), extraction for 1.5-2 h, standing at 8° C. for 24 h, and filtration to obtain a second filtrate and a second filter residue, where a weight ratio of the dried first filter residue to the ethanol solution was 1:(6-8) (preferably, 1:7). During the extraction, stirring was performed once every 10 min at a rate of 200-300 rpm.

The second filter residue was soaked with a 60% (v/v) ethanol solution for 6-7 h (preferably, 6.5 h) followed by heating to 65-75° C. (preferably, 70° C.), extraction for 2.5-3 h, standing at 8° C. for 24 h, and filtration to obtain a third filtrate and a third filter residue, where a weight ratio of the dried second filter residue to the ethanol solution was 1:(8-10) (preferably, 1:9). During the extraction, stirring was performed once every 10 min at a rate of 200-300 rpm. The first filtrate, the second filtrate, and the third filtrate were combined to obtain the Poria cocos extract, the mulberry leaf extract, or the hawthorn extract.
(S2) The oat dietary fiber powder, the konjac powder, the corn silk, the BMP powder, the soybean polypeptide powder, the mulberry leaf extract, the gardenia fruit oil, the cocoa powder, the Poria cocos extract, and the hawthorn extract were mixed uniformly according to a weight ratio in any one of Example 1-3 to obtain a mixture. The mixture was mixed with deionized water in a weight ratio of 1:(5-8) followed by stirring completely, heating to 45-65° C. (preferably, 55° C.), and decompressed concentration in vacuum to acquire a concentrated liquid.
(S3) The concentrated liquid was fed to a mixing container. Then the L-arabinose, the nutritional yeast, the pancreatin, and the xylitol was fed to the mixing container in a weight ratio in any one of Example 1-3 followed by stirring uniformly to obtain the polysaccharide-peptide composite.

Therefore, through the repeated extraction of mulberry leaves and hawthorn raw materials, the active ingredients (such as mulberry leaf polysaccharides, mulberry leaf alkaloids, mulberry flavonoids, hawthorn polysaccharides, etc.) could be fully released. In addition, the filter residue was re-extracted after the first extraction, which maximized the utilization rate of raw materials, so as to obtain higher purity of raw materials, ensuring that active ingredients could effectively exert hypoglycemic effects.

### Example 5

Provided herein was a polysaccharide-peptide composite, which was prepared from 20 parts by weight of oat dietary fiber powder, 11 parts by weight of konjac powder, 12 parts by weight of a corn silk, 20 parts by weight of a BMP powder, 10 parts by weight of soybean polypeptide powder, 6 parts by weight of a mulberry leaf extract, 6 parts by weight of a gardenia fruit oil, 5 parts by weight of cocoa powder, 5 parts by weight of L-arabinose, 3 parts by weight of a Poria cocos extract, 6 parts by weight of a hawthorn extract, 1 parts by weight of nutritional yeast, 2 parts by weight of a pancreatin, and 5 parts by weight of xylitol. The nutritional yeast included a selenium-rich yeast. The pancreatin included trypsin, amylopsin and pancreatic lipase with a weight ratio of 1:2:2.

The bitter melon polypeptide powder was prepared through the following steps.
(S1) A fresh bitter melon was soaked in deionized water at 25° C. for 10.5 h, where a weight ratio of the raw material to the deionized water was 1:5. After that, the fresh bitter melon was taken out followed by rinsing with deionized water for 2-3 times to remove pesticide residues, drug residues and impurities to obtain a rinsed bitter melon.
(S2) The rinsed bitter melon was subjected to drying, crushing and grinding to obtain a bitter melon slurry.
(S3) The bitter melon slurry was mixed with a buffer solution in a weight ratio of 1:4 to form a mixed system, where the buffer solution was a buffer system containing acid, alkali, salt and other reagents, i.e., phosphate buffer. The volume of the mixed system was recorded at this time as initial volume V₀. Then the mixed system was regulated to pH 6.8-7 followed by temperature treatment to obtain a bitter melon extract.

The temperature treatment was performed through the following steps.

The mixed system was subjected to heating to 50° C., keeping at 50° C. for 55 min, cooling to 22° C., and keeping at 22° C. for 28 min, and a volume of the mixed system was recorded at this time as a first volume V₁. During the aforementioned heating and insulation processes, the water and acids in the mixed system were evaporated, resulting in the change in the solubility of acid, base and inorganic ions in the mixed system. Therefore, after the aforementioned heating and insulation processes, a first mixed liquid was added to the mixed system for compensation such that the mixed system could constantly stay at a good extraction environment, where the first mixed liquid contained deionized water and the buffer solution with a weight ratio of 4:1; and a volume of the first mixed liquid was calculated by: (Vₜ-V₁)*0.6. After being added with the first mixed liquid, the mixed system was subjected to heating to 65° C., keeping at 65° C. for 65 min, cooling to 50° C., and keeping at 50° C. for 30 min, and a volume of the mixed system was recorded at this time as a second volume V₂. A second mixed liquid was added to the mixed system, where the second mixed liquid contained deionized water and the buffer solution with a weight ratio of 3:1; and a volume of the second mixed liquid was calculated by: (Vₜ-V₂)*0.75. In this case, the temperature of heating and insulation was higher, so the evaporation of water and acids in the mixed system was greater. As a consequence, the addition volume of the second mixed liquid was increased (to 75%), and the proportion of the buffer solution in the second mixed liquid was also increased. After being added with the second mixed liquid, the mixed system was subjected to heating to 85° C. for 80 min, and cooling to 70° C. for 40 min.

In this step, through periodic heating and insulation, cellular structure (such as cell wall) of components were repeatedly impacted and destroyed under different temperature environment. And at the same time, after heating and insulation, the reaction system was supplemented with corresponding proportion of water and the buffer solution for compensating the evaporation of water and acids, so that the reaction system was always in a great extraction environment to achieve the best extraction effect.
(S4) After the bitter melon extract was cooled to 20-25° C., enzymatic hydrolysis was performed on the bitter melon extract to obtain a polypeptide enzymatic hydrolysis product, where the enzymatic hydrolysis was performed through the following steps.

Primary Enzymatic Hydrolysis

The bitter melon extract was regulated to pH 7.0. Trypsin was added to the extract followed by heating to 37° C. under stirring at 80-100 r/min and keeping at 37° C. for 55 min to obtain a first enzymatic hydrolysis system, where the trypsin was 5% by weight of the extract.

Secondary Enzymatic Hydrolysis

After the first enzymatic hydrolysis system was cooled to 20-25° C., the first enzymatic hydrolysis system was regulated to pH 3.5. Pectinase was added to the first enzymatic hydrolysis system followed by heating to 50° C. under stirring at 80-100 r/min and keeping at 50° C. for 50 min to obtain a second enzymatic hydrolysis system, where the pectinase was 3% by weight of the first enzymatic hydrolysis system.

Tertiary Enzymatic Hydrolysis

After the second enzymatic hydrolysis system was cooled to 20-25° C., the second enzymatic hydrolysis system was regulated to pH 4.7. Cellulase was added to the second enzymatic hydrolysis system followed by heating to 58° C. under stirring at 80-100 r/min and keeping at 58° C. for 35 min to obtain an enzymatic hydrolysis product, where the cellulase was 2% by weight of the second enzymatic hydrolysis system.

As the components used herein were plant components, of which cellular structure contained cell walls. Therefore, in this step, through the use of different enzymes and enzymatic conditions under different stages for complete enzymatic hydrolysis of cell walls, cellulose and pectin of the cell walls were destroyed, which facilitated the full release of active ingredients (such as BMP protein) in the cell walls to enhance the extraction efficiency.
(S5) After the enzymatic hydrolysis was completed, the enzymatic hydrolysis product was heated to 90° C. for 10 min for inactivation to obtain a crude BMP extraction system. Activated carbon was added to the crude BMP extraction system, followed by uniform stirring, keeping at 65° C. for 75 min, centrifugation, and residue removal to obtain a first supernatant, where the activated carbon was 4-5% by weight of the crude BMP extraction system.

The first supernatant was filtered with diatomite at 0.25 MPa to obtain a first filtrate. The first filtrate was added with activated carbon followed by standing for 45-50 min, centrifugation, and residue removal to obtain a second supernatant, where the activated carbon was 4-5% by weight of the first filtrate.

Through the adsorption treatment by activated carbon and diatomite, the pigment, suspended particles and colloid in the enzymatic hydrolysate of BMP were removed to offer a final product with high purity.
(S6) The second supernatant was filtered with a ceramic microfiltration membrane with a pore size of 0.5-0.8 m at 60° C. to obtain a second filtrate, where the ceramic microfiltration membrane was used in parallel with three membranes.

The second filtrate was filtered with a spiral wound ultrafiltration membrane having a cut-off molecular weight of 100-200 kDa at 45-50° C. to remove macromolecular impurities to obtain a third filtrate, where the spiral wound ultrafiltration membrane was used in parallel with two membranes.

The third filtrate was concentrated with a spiral reverse-osmosis membrane with having a cut-off molecular weight of 150-1000 kDa below 40° C. to remove water, residual inorganic salts and small molecule impurities to obtain a BMP concentrate, where the solid content of the BMP concentrate was equal to or more than 40%; and the spiral reverse-osmosis membrane was a high-pressure concentration membrane, made of polysulfone (PS), polyethersulfone (PFS), or other composite material film, and used in parallel with four membranes.

In this step, the multilayer membrane separation and purification technology was used to separate and purify the BMP protein. The low concentration temperature used herein effectively guaranteed the natural activity and high content of the BMP protein.
(S7) The BMP concentrate was subjected to vacuum freeze drying to obtain the BMP powder, where the BMP powder contained 30% or more by weight of BMP.

Moreover, gardenia fruit was abundant, cheap, and had a variety of functions, and has played a more and more important role in the modern food industry. Specifically, gardenia fruit mainly contained flavonoids, iridoids, crocin, pectin, tannins, polysaccharides, crocetin, and volatile oil. Among them, flavonoids had an auxiliary therapeutic effect on hypertension and other diseases, and could reduce blood pressure and blood sugar. Hence, this example also provided a method for extracting the gardenia fruit oil, which included the following steps.
(S1) A fresh gardenia fruit was soaked in water at 25° C. for 24-36 h, followed by rinsing with water 2-3 times, drying, grinding, screening with a 100-mesh sieve to obtain a gardenia fruit powder.
(S2) The gardenia fruit powder was mixed with deionized water in a weight ratio of 1:8 to obtain a mixture, and the mixture was subjected to enzymatic hydrolysis to obtain an enzymatic hydrolysis product, where the enzymatic hydrolysis was performed through the following steps.

Primary Enzymatic Hydrolysis

The mixture was added with trypsin and a permeability-regulating fluid, where the trypsin was 5% by weight of the gardenia fruit powder; and the permeability-regulating fluid was 50% by weight of the gardenia fruit powder. The mixture was regulated to pH 7.0, followed by heating to 43.5° C. under stirring, and keeping at 43.5° C. for 30-45 min (preferably, 35 min) to obtain a first enzymatic hydrolysis system, where the permeability-regulating fluid consisted of an acid solution, glycerol, sodium chloride, and lysozyme in a weight ratio of 1:0.8:0.03:0.04; and the acid solution was a citric acid-sodium citrate buffer solution with a pH of 6-7.

Secondary Enzymatic Hydrolysis

After the first enzymatic hydrolysis system was cooled to 20-25° C., the first enzymatic hydrolysis system was regulated to pH 4.0. Pectinase was added to the first enzymatic hydrolysis system, followed by heating to 55° C. under stirring, and keeping at 55° C. for 32 min to obtain a second enzymatic hydrolysis system, where the pectinase was 4% by weight of the first enzymatic hydrolysis system.

Third Enzymatic Hydrolysis

After the second enzymatic hydrolysis system was cooled to 20-25° C., the second enzymatic hydrolysis system was regulated to pH 5.0. Cellulase was added to the second enzymatic hydrolysis system, followed by heating to 60° C. under stirring, and keeping at 50-65° C. for 30 min to obtain the enzymatic hydrolysis product, where the cellulase was 3.5% by weight of the second enzymatic hydrolysis system.
(S3) After the enzymatic hydrolysis was completed, the third enzymatic hydrolysis product was heated to 85° C. followed by keeping at 85° C. for 10 min for inactivation to obtain a gardenia fruit extraction enzymatic hydrolysis system.
(S4) The gardenia fruit enzymatic hydrolysis system was added with activated carbon followed by uniform stirring, keeping at 65° C. for 75 min, centrifugation, and residue removal to obtain a crude gardenia fruit oil extract, where the activated carbon was 4-5% by weight of the gardenia fruit enzymatic hydrolysis system. Then the crude gardenia fruit oil extract was filtered with diatomite at 0.35 MPa to obtain a filtrate. After that, the filtrate was added with activated carbon followed by standing for 45-50 min, centrifugation, and residue removal to obtain a supernatant, where the activated carbon was 3% by weight of the filtrate. The supernatant was subjected to standing for 2.5 h to collect an oil layer as the gardenia fruit oil.

Evaluation tests for lowering glycated hemoglobin (HbA1c) were described below.

Healthy male Sprague-Dawley (SD) rats with a body weight of 185-225 g were selected and fed adaptively for one week. After adaptive feeding, the SD rats were randomly divided into six groups each for 20 rats. One of the six groups was selected as a blank control group, and fed with ordinary feed. The rest five groups were subjected to intragastric administration with high-lipid emulsion for 10 mL/kg per day for 1 month. After the last feeding, rats were fasted for 12 h expect for water. The rats fed with high-lipid emulsion were subjected to disposable intraperitoneal injection with streptozotocin STZ solution for 25 mg/kg to establish a diabetic rat model. After streptozotocin injection for 72 h, the rats were fasted for 12 h expect for water and was taken blood samples from the tail to detect fasting blood sugar. The rat with a fasting blood glucose>10.0 mmol/L was considered as a successful model. The blank control group was intraperitoneally injected with citric acid-sodium citrate buffer solution.

The rats that were successfully modeled were randomly divided into model group, positive control group, low-dose group, medium-dose group and high-dose group according to blood sugar and body weight, with 20 rats in each group. The polysaccharide-peptide composite having functions of lowering blood sugar and glycated hemoglobin (denoted as polysaccharide-peptide composite) prepared in Examples 1-3 were diluted with water into intragastric solution, which were fed to the low-dose group for 1.5 g/kg, the medium-dose group for 2.5 g/kg and the high-dose group for 4.0 g/kg, respectively. While the positive control group was given metformin hydrochloride solution. Each group was given intragastric administration once per day for 8 weeks. Before detection, rats were fasted for 12 h expect for water. After the last feeding for 2 h, the urine was collected and tested for HbA1c content and blood lipid content. The results were shown in Table 3. The above groups were fed with different feeds and not restricted for drinking water.

TABLE 3
Comparison of effects of polysaccharide-peptide composite
on contents of HbA1c and blood lipid of rats

| | | Number | Content of | Content of blood |
|---|---|---|---|---|
| Groups | | of rat | HbA1c (ng/mL) | lipid (mmol/L) |
| Blank control group | | 20 | 26.54 ± 3.32 | 1.68 ± 0.25 |
| Model group | | 20 | 50.78 ± 3.76 | 3.29 ± 0.58* |
| Positive control group | | 20 | 35.14 ± 1.25 | 2.52 ± 0.24^{#}* |
| Low- | Example 1 | 20 | 46.69 ± 3.43 | 2.98 ± 0.48* |
| dose group | Example 2 | 20 | 47.30 ± 2.83 | 3.02 ± 0.34* |
| | Example 3 | 20 | 46.18 ± 2.03 | 3.14 ± 0.42* |
| Medium- | Example 1 | 20 | 38.17 ± 0.28 | 2.89 ± 0.58* |
| dose group | Example 2 | 20 | 36.24 ± 3.55 | 2.87 ± 0.61* |
| | Example 3 | 20 | 38.53 ± 1.20 | 2.92 ± 0.63* |
| High- | Example 1 | 20 | 30.80 ± 1.59 | 2.87 ± 0.47* |
| dose group | Example 2 | 20 | 30.14 ± 2.27 | 2.80 ± 0.49* |
| | Example 3 | 20 | 30.48 ± 3.09 | 2.71 ± 0.53* |

Note:
Compared with blank control group,
** P < 0.01; and compared with model group,
^{#}P < 0.05,
^{##} P < 0.01.

As shown in Table 3, compared with the blank control group, the contents of blood lipid and HbA1c in the model group were significantly increased (both increased by 50%), indicating the successful modeling of diabetic rats. Compared with model group, the contents of blood lipid and HbA1c in the positive control group and drug groups (high, medium and low-dose groups) were decreased, and presented significant differences among them. Specifically, the contents of HbA1c and blood lipid in high-dose group declined more obvious (about 40% and 18%, respectively), indicating that the polysaccharide-peptide composite prepared in this application had good therapeutic effect on diabetes.

Evaluation tests for lowering blood sugar were described below.

100 of healthy male rats were selected, numbered and fed adaptively in normal environment for 2 weeks, and free to eat and drink.

After adaptive feeding, the rats were randomly divided into 5 groups with 20 rats in each group. Only one group was selected as the blank control group, the rest groups were subjected to disposable intraperitoneal injection with 5% streptozotocin STZ solution for 200 mg/kg. If there were coma among those rats within 2 h, glucose sugar solution was fed until awake. Two days later, the fasting blood sugar was detected. The rat with a blood sugar of more than 10 was selected as a diabetic animal model.

The rats that were successfully modeled were randomly divided into model group, positive control group, low-dose group, medium-dose group and high-dose group according to blood sugar and body weight, with 20 rats in each group. The polysaccharide-peptide composites prepared in Examples 1-3 were diluted with water into intragastric solutions, which were fed to the low-dose group for 1.0 g/kg, the medium-dose group for 2.0 g/kg and the high-dose group for 3.0 g/kg, respectively. While the positive control group was given metformin hydrochloride solution. Each group was given intragastric administration once per day for 15 days. After that, rats were fasted for 8 h to measure fasting blood sugar and urine sugar. The results were shown in Table 4. The above groups were fed with different feeds and not restricted for drinking water.

During the experiment, rats of each group were intraperitoneally injected every day for 15 consecutive days. After 45 days, rats were fasted for 8 h to measure fasting blood sugar. At the end of the experiment, the final blood sugar content was recorded.

TABLE 4
Comparison of effects of polysaccharide-peptide composite
on contents of urine sugar and fasting blood sugar of rats

| | | | Content of | Content of |
|---|---|---|---|---|
| | | The number | urine sugar | fasting blood |
| Groups | | of rats | (mmol/L) | sugar (mmol/L) |
| Blank control group | | 20 | 0.60 ± 0.01 | 5.31 ± 0.53 |
| Model group | | 20 | 1.74 ± 0.56 | 14.86 ± 2.38 |
| Positive control group | | 20 | 1.44 ± 0.23 | 8.55 ± 1.37 |
| Low- | Example 1 | 20 | 1.54 ± 0.09 | 11.78 ± 2.64 |
| dose group | Example 2 | 20 | 1.63 ± 0.83 | 11.72 ± 2.97 |
| | Example 3 | 20 | 1.59 ± 0.45 | 11.69 ± 2.37 |
| Medium- | Example 1 | 20 | 1.21 ± 0.12 | 9.17 ± 0.96 |
| dose group | Example 2 | 20 | 1.18 ± 0.02 | 9.15 ± 0.44 |
| | Example 3 | 20 | 1.16 ± 0.37 | 9.03 ± 1.12 |
| High- | Example 1 | 20 | 0.78 ± 0.11 | 7.42 ± 1.25 |
| dose group | Example 2 | 20 | 0.75 ± 0.77 | 7.13 ± 1.32 |
| | Example 3 | 20 | 0.74 ± 0.69 | 6.84 ± 1.41 |

Table 4 showed that compared with the model group, the contents of urine sugar and fasting blood sugar of the rats in the positive control group and drug groups (high, medium and low-dose group) declined, and presented significant differences among them. Specifically, compared with the model group, the contents of urine sugar in the high, medium and low-dose groups decreased by 9%, 32% and 56%, respectively, and the content of fasting blood sugar decreased by 21%, 39% and 52%, respectively, indicating that high-dose polysaccharide-peptide composite significantly reduced contents of urine sugar and fasting blood sugar.

Evaluation tests for lowering blood lipid were described below.

60 of healthy male rats were selected, numbered and fed adaptively in normal environment for 2 weeks, and free to eat and drink. After the adaptive feeding, the rats were randomly divided into 6 groups with 10 rats in each group, consisting of a blank control group, a high-lipid model group, a positive treatment group, a high-dose group, a medium-dose group and a low-dose group. Except the blank control group, the other groups were given high lipid diet. The polysaccharide-peptide composites prepared in this application and simvastatin were respectively diluted. The positive control group was given simvastatin solution by intragastric administration for 50 mg/kg bw d, and the high, medium and low-dose groups were given polysaccharide-peptide solutions for 100 mg/kg bw d, 50 mg/kg bw d and 25 mg/kg bw d, respectively. Other groups were intragastric with corresponding volume of distilled water. The treatment period lasted for 4 weeks. After continuous feeding for 10 weeks, and after the last feeding, rats were fasted excepted for water for 10 h, and total cholesterol (TC) and triglyceride (TG) were measured by aortic blood samples, and the results were shown in Table 5.

TABLE 5
Comparison of effects of polysaccharide-peptide
composite on contents of TC and TG of rats

| | | The number | Content of | Content of |
|---|---|---|---|---|
| Groups | | of rats | TC (mmol/L) | TG (mmol/L) |
| Blank control group | | 10 | 1.79 ± 0.25 | 0.42 ± 0.18 |
| Model group | | 10 | 3.41 ± 0.58* | 0.57 ± 0.18 |
| Positive control group | | 10 | 2.59 ± 0.24^{#}* | 0.32 ± 0.14^{#} |
| Low- | Example 1 | 10 | 3.02 ± 0.48* | 0.46 ± 0.14^{##} |
| dose group | Example 2 | 10 | 3.11 ± 0.34* | 0.45 ± 0.17^{##} |
| | Example 3 | 10 | 3.13 ± 0.42* | 0.41 ± 0.13^{##} |
| Medium- | Example 1 | 10 | 2.95 ± 0.58* | 0.41 ± 0.07^{##} |
| dose group | Example 2 | 10 | 2.94 ± 0.61* | 0.43 ± 0.09^{##} |
| | Example 3 | 10 | 2.96 ± 0.63* | 0.39 ± 0.06^{##} |
| High- | Example 1 | 10 | 2.88 ± 0.47* | 0.39 ± 0.06^{##} |
| dose group | Example 2 | 10 | 2.79 ± 0.49* | 0.38 ± 0.04^{##} |
| | Example 3 | 10 | 2.69 ± 0.53* | 0.36 ± 0.07^{##} |

Note:
Compared with the blank group,
*P < 0.05; and compared with the high-lipid group,
^{#}P < 0.05,
^{##}P < 0.01.

As can be seen from Table 5, compared with the high-lipid group, the contents of TC and TG of rats in the drug groups (high-dose, medium-dose and low-dose groups) were significantly decreased, proving that the polysaccharide-peptide composite prepared in this application had a good effect on lowering blood lipid.

It should be noted that the technical solutions in the above Examples 1-5 can be combined arbitrarily, and the technical solutions obtained after combination shall fall within the protection scope of this application.

In summary, through the extraction process including periodic warming, repeated digestion and multiple filtering, the content of bitter melon peptide-protein in the bitter melon peptide is equal to or greater than 30%. Moreover, bitter melon peptide is used in conjunction with other components, showing a significant effect in view of lowering blood sugar and glycated hemoglobin, and allowing users to get rid of side effects brought by the use of chemical hypoglycemic drugs.

The number of devices and the scale of processing shown herein are intended to simplify the description of the present disclosure. The application, modification and variation of the present disclosure are obvious to those skilled in the art.

Described above are merely illustrative of the disclosure, and are not intended to limit the disclosure. It should be understood that any modifications, replacements and variations made by those skilled in the art without departing from the spirit and scope of the disclosure should fall within the scope of the disclosure defined by the appended claims.

What is claimed is:

1. A polysaccharide-peptide composite, wherein the polysaccharide-peptide composite is prepared from 20-25 parts by weight of oat dietary fiber powder, 10-15 parts by weight of konjac powder, 10-15 parts by weight of a corn silk, 20-30 parts by weight of bitter melon peptide (BMP) powder, 10-12 parts by weight of soybean polypeptide powder, 5-10 parts by weight of a mulberry leaf extract, 5-10 parts by weight of a gardenia fruit oil, 5-10 parts by weight of cocoa powder, 5-10 parts by weight of L-arabinose, 3-5 parts by weight of a Poria cocos extract, 5-10 parts by weight of a hawthorn extract, 1-2 parts by weight of nutritional yeast, 2-5 parts by weight of a pancreatin, and 5-8 parts by weight of xylitol.

2. The polysaccharide-peptide composite of claim 1, wherein the nutritional yeast is selenium-rich yeast, chromium-rich yeast, or a combination thereof.

3. The polysaccharide-peptide composite of claim 1, wherein the pancreatin comprises trypsin, amylopsin, and pancreatic lipase with a weight ratio of 1:2:2.

4. The polysaccharide-peptide composite of claim 1, wherein the BMP powder is prepared through steps of:
(S11) soaking a raw material with deionized water at 25° C. for 10-12 h, wherein a weight ratio of the raw material to the deionized water is 1:5, and the raw material is fresh bitter melon, dried bitter melon, bitter melon seeds, or a combination thereof; and taking the raw material out followed by rinsing with deionized water 2-3 times;
(S2) subjecting the raw material to drying, crushing and grinding to obtain a bitter melon slurry;
(S3) mixing the bitter melon slurry with a buffer solution in a weight ratio of 1:(3-5) to form a mixed system; recording a volume of the mixed system as initial Volume V₀; and regulating the mixed system to pH 6.8-7 followed by temperature treatment to obtain a bitter melon extract;
wherein the temperature treatment is performed through steps of:
(S31) heating the mixed system to 45-55° C. and keeping the mixed system at 45-55° C. for 45-60 min; cooling the mixed system to 20-25° C., and keeping the mixed system at 20-25° C. for 25-30 min; and recording a volume of the mixed system at this time as a first volume V₁;
(S32) adding a first mixed liquid to the mixed system, wherein the first mixed liquid comprises deionized water and the buffer solution with a weight ratio of 4:1, and a volume of the first mixed liquid is calculated by: (V₀-V₁)*0.6; heating the mixed system to 60-75° C., and keeping the mixed system at 60-75° C. for 60-75 min; cooling the mixed system to 45-55° C., and keeping the mixed system at 45-55° C. for 30-35 min; and recording a volume of the mixed system at this time as second volume V₂; and
(S33) adding a second mixed liquid to the mixed system, wherein second mixed liquid comprises deionized water and the buffer solution with a weight ratio of 3:1, and a volume of the second mixed liquid is calculated by: (V₀-V₂)*0.75; heating the mixed system to 80-90° C., and keeping the mixed system at 80-90° C. for 75-85 min; and cooling the mixed system to 60-75° C. and keeping the mixed system at 60-75° C. for 35-45 min;
(S4) cooling the bitter melon extract to 20-25° C. followed by enzymatic hydrolysis to obtain an enzymatic hydrolysis product, wherein the enzymatic hydrolysis is performed through steps of:
regulating the bitter melon extract to pH 7.5-8.5; adding trypsin to the bitter melon extract followed by heating to 35-40° C. under stirring at 80-100 rpm and keeping at 35-40° C. for 45-60 min to obtain a first enzymatic hydrolysis system, wherein the trypsin is 5% by weight of the bitter melon extract;
cooling the first enzymatic hydrolysis system to 20-25° C., and adjusting the first enzymatic hydrolysis system to pH 3.0-4.0; adding pectinase to the first enzymatic hydrolysis system followed by heating to 45-55° C. under stirring at 80-100 rpm and keeping at 45-55° C. for 40-60 min to obtain a second enzymatic hydrolysis system, wherein the pectinase is 3% by weight of the first enzymatic hydrolysis system; and
cooling the second enzymatic hydrolysis system to 20-25° C., and regulating the second enzymatic hydrolysis system to pH 4.5-5.0; adding a cellulase to the second enzymatic hydrolysis system followed by heating to 55-60° C. under stirring at 80-100 rpm and keeping at 55-60° C. for 30-45 min to obtain the enzymatic hydrolysis product, wherein the cellulase is 2% by weight of the second enzymatic hydrolysis system;
(S5) heating the enzymatic hydrolysis product to 90° C. followed by keeping at 90° C. for 10 min for inactivation, so as to obtain a crude BMP extraction system; adding activated carbon to the crude BMP extraction system followed by uniform stirring, wherein the activated carbon is 4-5% by weight of the crude BMP extraction system; and keeping the crude BMP extraction system at 65° C. for 60-90 min followed by centrifugation to obtain a first supernatant; and
filtering the first supernatant with diatomite at 0.2-0.3 MPa to obtain a first filtrate; and adding activated carbon to the first filtrate followed by standing for 45-50 min and centrifugation to obtain a second supernatant, wherein the activated carbon is 4-5% by weight of the first filtrate;
(S6) filtering the second supernatant with a ceramic microfiltration membrane with a pore size of 0.5-0.8 m at 55-65° C. to obtain a second filtrate;
filtering the second filtrate with a spiral wound ultrafiltration membrane having a cut-off molecular weight of 100-200 kDa at 45-50° C. to obtain a third filtrate; and
concentrating the third filtrate with a spiral-wound reverse-osmosis membrane at 35-40° C. to remove water, residual inorganic salts and small molecule impurities to obtain a BMP concentrate; and
(S7) subjecting the BMP concentrate to vacuum freeze drying to obtain the BMP powder, wherein the BMP powder comprises 30% or more by weight of BMP.

5. The polysaccharide-peptide composite of claim 4, wherein the buffer solution is a phosphate buffer solution prepared from disodium hydrogen phosphate dodecahydrate and sodium dihydrogen phosphate dihydrate.

6. The polysaccharide-peptide composite of claim 1, wherein the gardenia fruit oil is extracted through steps of:
(S1) soaking a fresh gardenia fruit in water at 25° C. for 24-36 h, followed by rinsing with water 2-3 times, drying, grinding, screening with a 100-mesh sieve to obtain a gardenia fruit powder;
(S2) mixing the gardenia fruit powder with deionized water in a weight ratio of 1:(5-10) to obtain a mixture; and subjecting the mixture to enzymatic hydrolysis to obtain an enzymatic hydrolysis product, wherein the enzymatic hydrolysis is performed through steps of:
adding trypsin and a permeability-regulating fluid to the mixture, wherein the trypsin is 5% by weight of the gardenia fruit powder, and the permeability-regulating fluid is 45-55% by weight of the gardenia fruit powder; and adjusting the mixture to pH 6.5-7.5, followed by heating to 42-45° C. under stirring, and keeping at 42-45° C. for 30-45 min to obtain a first enzymatic hydrolysis system, wherein the permeability-regulating fluid consists of an acid solution, glycerol, sodium chloride, and lysozyme in a weight ratio of 1:(0.7-1.0):(0.02-0.05):(0.03-0.06);
cooling the first enzymatic hydrolysis system to 20-25° C., regulating the first enzymatic hydrolysis system to pH 3.5-4.5; and adding pectinase to the first enzymatic hydrolysis system followed by heating to 50-60° C. under stirring, and keeping at 50-60° C. for 30-35 min to obtain a second enzymatic hydrolysis system, wherein the pectinase is 4% by weight of the first enzymatic hydrolysis system; and
cooling the second enzymatic hydrolysis system to 20-25° C., and regulating the second enzymatic hydrolysis system to pH 4.0-5.5; and adding cellulase to the second enzymatic hydrolysis system followed by heating to 50-65° C. under stirring, and keeping at 50-65° C. for 25-35 min to obtain the enzymatic hydrolysis product, wherein the cellulase is 3.5% by weight of the second enzymatic hydrolysis system;
(S3) heating the enzymatic hydrolysis product to 85° C. followed by keeping at 85° C. for 10 min for inactivation to obtain a gardenia fruit extraction system; and
(S4) adding activated carbon to the gardenia fruit extraction system followed by uniform stirring, keeping at 65° C. for 65-85 min and centrifugation to obtain a crude gardenia fruit oil extract, wherein the activated carbon is 3% by weight of the gardenia fruit extraction system; filtering the crude gardenia fruit oil extract with diatomite at 0.3-0.4 MPa to obtain a filtrate; adding activated carbon to the filtrate followed by standing for 45-50 min and centrifugation to obtain a supernatant, wherein the activated carbon is 3% by weight of the filtrate; and subjecting the supernatant to standing for 2-3 h to collect an oil layer as the gardenia fruit oil.

7. A method of preparing the polysaccharide-peptide composite of claim 1, comprising:
(S1) preparing the BMP powder, the gardenia fruit oil, the Poria cocos extract, the mulberry leaf extract and the hawthorn extract, wherein the Poria cocos extract, the mulberry leaf extract or the hawthorn extract is prepared through steps of:
soaking a raw material in water for 12-15 h followed by boiling for 1-2 h and filtration to obtain a first filtrate and a first filter residue, wherein a weight ratio of the raw material to the water is 1:(8-10);
drying the first filter residue; soaking a dried first filter residue with a 60% (v/v) ethanol solution for 1-2 h followed by heating to 65-75° C., extraction for 1.5-2 h, standing at 8° C. for 24 h, and filtration to obtain a second filtrate and a second filter residue, wherein a weight ratio of the dried first filter residue to the ethanol solution is 1:(6-8); and during the extraction, stirring is performed once every 10 min with a stirring rate of 200-300 rpm; and
soaking the second filter residue with a 60% (v/v) ethanol solution for 6-7 h followed by heating to 65-75° C., extraction for 2.5-3 h, standing at 8° C. for 24 h, and filtration to obtain a third filtrate and a third filter residue, wherein a weight ratio of the second filter residue to the ethanol solution is 1:(8-10); and during the extraction, stirring is performed once every 10 min with a stirring rate of 200-300 rpm; and combining the first filtrate, the second filtrate, and the third filtrate to obtain the Poria cocos extract, the mulberry leaf extract, or the hawthorn extract;
(S2) uniformly mixing 20-25 parts by weight of the oat dietary fiber powder, 10-15 parts by weight of the konjac powder, 10-15 parts by weight of the corn silk, 20-30 parts by weight of the BMP powder, 10-12 parts by weight of the soybean polypeptide powder, 5-10 parts by weight of the mulberry leaf extract, 5-10 parts by weight of the gardenia fruit oil, 5-10 parts by weight of the cocoa powder, 3-5 parts by weight of the Poria cocos extract, and 5-10 parts by weight of the hawthorn extract to obtain a mixture; mixing the mixture with deionized water in a weight ratio of 1:(5-8) followed by stirring, heating to 45-65° C., and vacuum concentration to obtain a concentrate; and
(S3) mixing the concentrate with 5-10 parts by weight of the L-arabinose, 1-2 parts by weight of the nutritional yeast, 2-5 parts by weight of the pancreatin, and 5-8 parts by weight of the xylitol followed by stirring to obtain the polysaccharide-peptide composite.

8. A method for lowering blood sugar and lipid and glycosylated hemoglobin in a subject in need thereof, comprising:
administering the polysaccharide-peptide composite of claim 1 to the subject.

9. The method of claim 8, wherein the polysaccharide-peptide composite is administered orally at a dose of 30-50 mg/(kg·d).
